(19) United States
(12) Patent Application Publication
(10) Pub. No.: US 2021/0236759 A1
(43) Pub. Date: Aug. 5, 2021

(54) Continuous Positive Airway Pressure Machine Cover Assembly

(71) Applicant: Arlene Bryant- Hill, De Soto, TX (US)

(72) Inventor: Arlene Bryant- Hill, De Soto, TX (US)

(21) Appl. No.: 16/781,714

(22) Filed: Feb. 4, 2020

(51) Int. Cl. A61M 16/06

(52) U.S. Cl. CPC A61M 16/06; A61M 2205/0216; A61M 2209/06

(57) ABSTRACT

A CPAP cover assembly includes a tube that has a front end and a back end. Each of the back end and the front end is open into an interior of the tube thereby facilitating the tube to insertably receive a CPAP mask. A front elastomeric band is coupled to the tube and the front elastomeric band is aligned with the front end for biasing the front end into a substantially closed position. In this way the front elastomeric band retains the tube around the CPAP mask. A back elastomeric band is coupled to the tube and the back elastomeric band is aligned with the back end for biasing the back end into a substantially closed position. It this way the back elastomeric band retains the tube around the CPAP mask.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to cover devices and more particularly pertains to a new cover device for storing a continuous positive airway pressure (CPAP) machine mask when the CPAP mask is not in use.

### (2) Description of Related Art including information disclosed under 37 CFR 1.97 and 1.98

The prior art relates to cover devices. The prior art discloses a variety of CPAP masks, each having novel structural elements and functions, for CPAP therapy. The prior art discloses a disposable envelope that is foldable around a CPAP mask for storage. The prior art also discloses a CPAP bag for storing a CPAP mask, and the CPAP bag includes a window for viewing the CPAP mask within the CPAP bag. Additionally, the prior art disclose a plurality of liners that are positionable between a CPAP mask and a user's skin for enhancing comfort for the user.

### BRIEF SUMMARY OF THE INVENTION

An embodiment of the disclosure meets the needs presented above by generally comprising a tube that has a front end and a back end. Each of the back end and the front end is open into an interior of the tube thereby facilitating the tube to insertably receive a CPAP mask. A front elastomeric band is coupled to the tube and the front elastomeric band is aligned with the front end for biasing the front end into a substantially closed position. In this way the front elastomeric band retains the tube around the CPAP mask. A back elastomeric band is coupled to the tube and the back elastomeric band is aligned with the back end for biasing the back end into a substantially closed position. It this way the back elastomeric band retains the tube around the CPAP mask.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a CPAP cover assembly according to an embodiment of the disclosure.

FIG. 2 is a top view of an embodiment of the disclosure.

FIG. 3 is a front view of an embodiment of the disclosure.

FIG. 4 is a phantom in-use view of an embodiment of the disclosure.

### DETAILED DESCRIPTION OF THE INVENTION

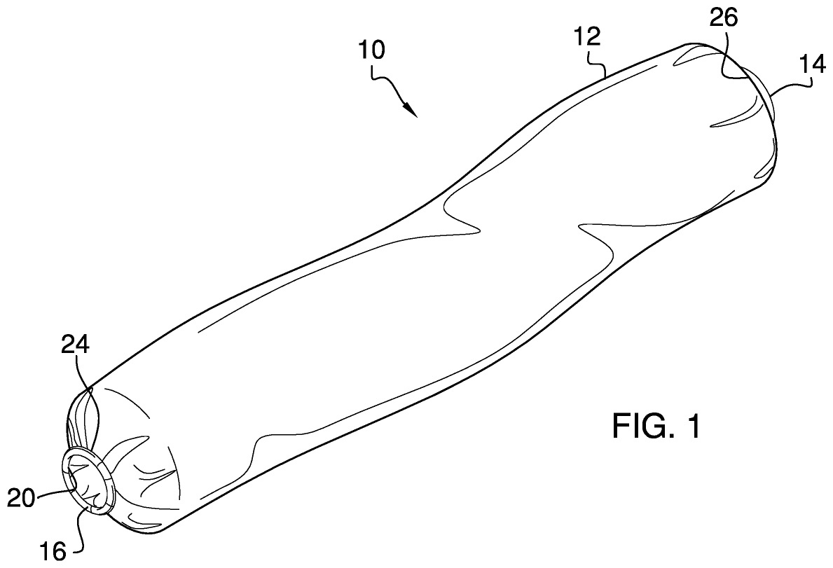

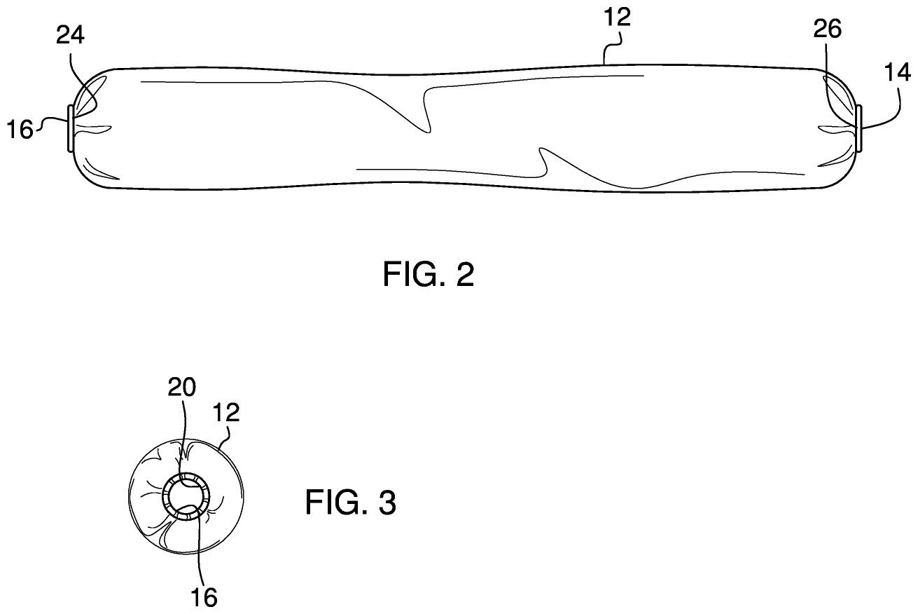

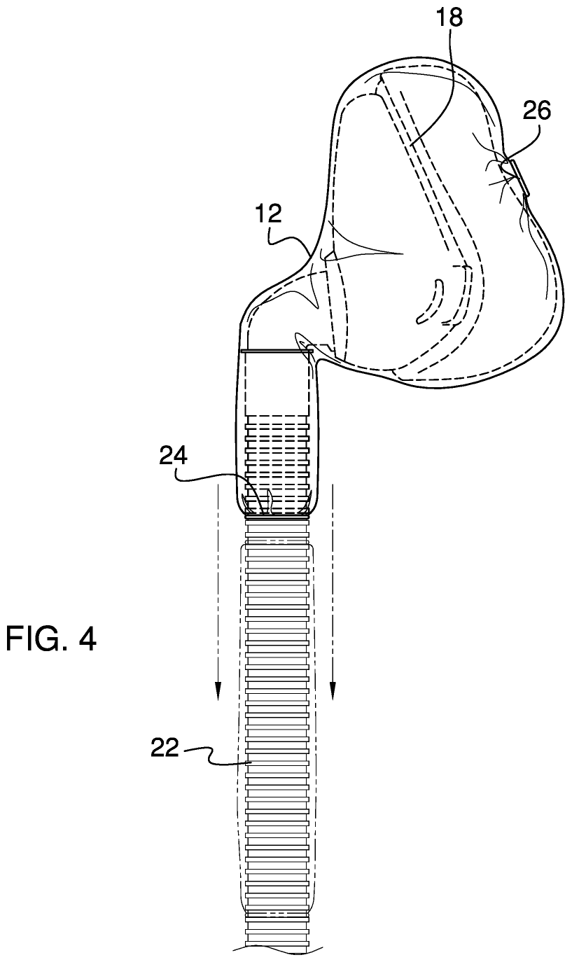

With reference now to the drawings, and in particular to FIGS. 1 through 4 thereof, a new cover device embodying the principles and concepts of an embodiment of the disclosure and generally designated by the reference numeral 10 will be described.

As best illustrated in FIGS. 1 through 4, the CPAP cover assembly 10 generally comprises a tube 12 that has a back end 14 and a front end 16. Each of the front end 16 and the back end 14 is open into an interior of the tube 12 to insertably receive a CPAP mask 18. Additionally, each of the front end 16 and the back end 14 has a bounding edge 20. The tube 12 is elongated between the back end 14 and the front end 16, and the tube 12 has a length of at least 8.0 inches. In this way the tube 12 can completely contain the CPAP mask 18 and a portion of an air supply hose 22 of the CPAP mask 18. Moreover, the tube 12 is comprised of a fluid impermeable material to protect the CPAP mask 18 from moisture, bacteria and dust.

A front elastomeric band 24 is provided and the front elastomeric band 24 is coupled to the tube 12. The front elastomeric band 24 is aligned with the front end 16 for biasing the front end 16 into a substantially closed position. In this way the front elastomeric band 24 retains the tube 12 around the CPAP mask 18. The front elastomeric band 24 is integrated into the tube 12 and the elastomeric band 24 is coextensive with the bounding edge 20 of the front end 16.

A back elastomeric band 26 is provided and the back elastomeric band 26 is coupled to the tube 12. The back elastomeric band 26 is aligned with the back end 16 for biasing the back end 16 into a substantially closed position. In this way the back elastomeric band 26 retains the tube 12 around the CPAP mask 18. The back elastomeric band 26 is integrated into the tube 12 and the elastomeric band 24 is coextensive with the bounding edge 20 of the back end 16.

In use, the CPAP mask 18 is inserted into the front end 16 of the tube 12 to containing the CPAP mask 18 and a portion of the air supply hose 22 in the tube 12. In this way the CPAP mask 18 is stored in the tube 12 not being used. Thus, the tube protects the CPAP mask 18 from moisture, bacteria, dust and other contaminants while the CPAP mask 18 is stored in the tube 12. The CPAP mask 18 is removed from the tube 12 prior to wearing the CPAP mask 18.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A CPAP cover assembly for covering a CPAP mask and associated hardware when the CPAP mask is not in use, said assembly comprising:
a tube having a front end and a back end, each of said back end and said front end being open into an interior of said tube wherein said tube is configured to insertably receive a CPAP mask;
a front elastomeric band being coupled to said tube, said front elastomeric band being aligned with said front end for biasing said front end into a substantially closed position wherein said front elastomeric band is configured to retain said tube around the CPAP mask; and
a back elastomeric band being coupled to said tube, said back elastomeric band being aligned with said back end for biasing said back end into a substantially closed position wherein said back elastomeric band is configured to retain said tube around the CPAP mask.

2. The assembly according to claim 1, wherein said tube has a length of at least 8.0 inches wherein said tube is configured to completely contain the CPAP mask and a portion of an air supply hose of the CPAP mask.

3. The assembly according to claim 1, wherein said tube is elongated between said front end and said back end, each of said back end and said front end having a bounding edge, said tube being comprised of a fluid impermeable material wherein said tube is configured to protect the CPAP mask from moisture, bacteria and dust.

4. The assembly according to claim 3, wherein said front elastomeric band is integrated into said tube, said front elastomeric band being coextensive with said bounding edge of said front end.

5. The assembly according to claim 3, wherein said back elastomeric band is integrated into said tube, said back elastomeric band being coextensive with said bounding edge of said back end.

6. A CPAP cover assembly for covering a CPAP mask and associated hardware when the CPAP mask is not in use, said assembly comprising:
a tube having a front end and a back end, said back end being open into an interior of said tube wherein said back end is configured to insertably receive a CPAP mask, said front end being closed wherein said tube is configured to contain the CPAP mask, said tube being elongated between said front end and said back end, said tube having a length of at least 8.0 inches wherein said tube is configured to completely contain the CPAP mask and a portion of an air supply hose of the CPAP mask, said back end having a bounding edge, said tube being comprised of a fluid impermeable material wherein said tube is configured to protect the CPAP mask from moisture, bacteria and dust;
a front elastomeric band being coupled to said tube, said front elastomeric band being aligned with said front end for biasing said front end into a substantially closed position wherein said front elastomeric band is configured to retain said tube around the CPAP mask, said front elastomeric band being integrated into said tube, said front elastomeric band being coextensive with said bounding edge of said front end; and
a back elastomeric band being coupled to said tube, said back elastomeric band being aligned with said back end for biasing said back end into a substantially closed position wherein said back elastomeric band is configured to retain said tube around the CPAP mask, said back elastomeric band being integrated into said tube, said back elastomeric band being coextensive with said bounding edge of said back end.

6. A CPAP cover system for covering a CPAP mask and associated hardware when the CPAP mask is not in use, said system comprising:
a CPAP mask having a body and an air supply hose being fluidly coupled thereto wherein said CPAP mask is configured to be worn on a user's face for CPAP therapy;
a tube having a front end and a back end, said back end being open into an interior of said tube to insertably receive said CPAP mask, said front end being closed to contain said CPAP mask, said tube being elongated between said front end and said back end, said tube having a length of at least 8.0 inches where to completely contain said CPAP mask and a portion of said air supply hose, said back end having a bounding edge, said tube being comprised of a fluid impermeable material wherein said tube is configured to protect said CPAP mask from moisture, bacteria and dust;
a front elastomeric band being coupled to said tube, said front elastomeric band being aligned with said front end for biasing said front end into a substantially closed position wherein said front elastomeric band is configured to retain said tube around the CPAP mask, said front elastomeric band being integrated into said tube, said front elastomeric band being coextensive with said bounding edge of said front end; and
a back elastomeric band being coupled to said tube, said back elastomeric band being aligned with said back end for biasing said back end into a substantially closed position wherein said back elastomeric band is configured to retain said tube around the CPAP mask, said back elastomeric band being integrated into said tube, said back elastomeric band being coextensive with said bounding edge of said back end.
